(19) United States
(12) Patent Application Publication
(10) Pub. No.: US 2007/0100246 A1
(43) Pub. Date: May 3, 2007

(54) Heart rate based bioassessment method and apparatus

(76) Inventor: Christopher Thomas Hyde, Elkton, MD (US)

Correspondence Address: BACON & THOMAS, PLLC, 625 SLATERS LANE, FOURTH FLOOR, ALEXANDRIA, VA, 22314, US

(21) Appl. No.: 11/589,834

(22) Filed: Oct. 31, 2006

Related U.S. Application Data
(60) Provisional application No. 60/731,679, filed on Oct. 31, 2005.

(51) Int. Cl. A61B 5/04

(52) U.S. Cl. 600/509

(57) ABSTRACT

The physiological or mental functioning of a patient is assessed by subjecting a patient to a protocol expected to activate or suppress the neurhormonal systems that regulate blood flow. A physiological signal indicative of the cardiac cycle, such as the electrocardiograph signal, is then monitored in synchrony with performance of the protocol. The physiological signal is processed to quantify the low frequency oscillation during different conditions during the protocol, and the results are interpreted in terms of cardiovascular changes, such as changes in blood flow to active tissues in the body. The results are then compared to normative data to assess the physiological or mental functioning of the patent.

This application claims priority of U.S. Provisional Patent Application No. 60/731,679, filed Oct. 31, 2005.

### BACKGROUND OF THE INVENTION

1. Field of the Invention

The invention relates to a method and apparatus for measuring and analyzing physiological data for the purpose of evaluating the human body's response to various situations. The physiological data can be used to identify individuals who have biological weaknesses or abnormalities.

More specifically, this invention relates to a method and apparatus for quantitatively assessing cardiovascular changes in response to specific conditions. Many of the human body's responses to various situations require changes in blood flow. For example, increased muscular activity is supported by increased heart rate and cardiac output. As a second example, mental activity is supported by increased blood flow to the active region of the brain. Thus, measuring these changes in blood flow can provide a more convenient and noninvasive means of monitoring physiological activities that are difficult to measure (e.g., mental activity).

The present invention is based on the principle that specific characteristics of the cardiac cycle (or other physiological measures) reflect sustained changes in blood flow. When this physiological characteristic is measured during specific conditions, it reflects the extent to which an individual responded to the conditions with a change in blood flow to a tissue or an organ. The person's physiological response can be characterized as healthy or abnormal by comparing his/her results to results of healthy individuals. The Inventor has discovered that this procedure can best be accomplished by analysis of the frequency spectrum of the heart rate time series while individuals perform a specific predetermined protocol; the protocols are designed to elicit specific physiological activities relevant to the disease of concern.

2. Description of Related Art

It has been known for some time that the body shifts blood flow to active tissues (i.e., parts of organs) when these tissues are actively performing required physiological function; increased tissue activity requires increased blood flow to support the increased work of the tissue. For example, blood flow increases in the specific areas of the brain which are involved in performing mental work, blood flow increases to the muscles during exercise, and blood flow increases to the gastrointestinal tract after meals. Changes in blood flow are achieved through a combination of vasodilation (more blood flow to the active tissues) and vasoconstriction (less blood flow to other tissues). In instances when the changes in blood flow must be maintained for prolonged periods of time, two neurohormonal systems play significant roles: the angiotensin-renin-aldosterone system (RAAS) and the sympathetic nervous system (SNS). Other systems are also involved in controlling blood flow to specific tissues within the body.

Changes in heart rate over time, or heart rate variability (HRV), have often been used as a measure of cardiovascular functioning. For example, heart rate variability has been used as a predictor of morbidity and mortality in patients after heart attacks (Kleiger, R. E., J. P. Miller, et al. (1987). “The Multicenter Post-Research Group: Decreased heart rate variability and its association with increased mortality after acute myocardial infarction.” American Journal of Cardiology 59: 256-262. And Nolan, J., P. D. Batin, et al. (1998). “Prospective study of heart rate variability and mortality in chronic heart failure: results of the United Kingdom heart failure evaluation and assessment of risk trial (UK-heart).” Circulation 98(15): 1510-6.). Sophisticated heart rate analyses, such as the interbeat or R-R interval analysis described in U.S. Pat. No. 6,480,733, provide a general indication of potential for heart failure. However, these methods measure HRV during only one condition; they do not relate the HRV with a specific physiological process (i.e., regional changes in blood flow). To date, little attention has been paid to the possibility of using heart rate to assess neurohormonal activity in response to specific physiological or psychological conditions.

Heart rate changes from beat to beat; these increases and decreases are not random, but cyclical, in nature. Much research and several inventions have focused on the applications of heart rate variability (HRV) which is a measure of total, overall variability of instantaneous heart rate. Spectral analysis is group of methods for analyzing heart rate variability that separates heart rate variability into several categories based on the frequency of the beat-to-beat changes in heart rate. Fast Fourier transforms are one of the spectral analysis methods.

The normally occurring fluctuations in heart rate can be divided into several bands based on the frequency of the fluctuations. Frequency bands refer to the cyclic patterns of increasing and decreasing heart rate at a certain oscillation. Several cardiac oscillations occur simultaneously, but each one can be distinguished based on its period, or length, of the heart rate cycle. For example, the average cycle of the fast cardiac rhythm, respiratory sinus arrhythmia (RSA), has a duration of approximately three or four seconds, which occurs at the frequency of respiration. In contrast, the complete cycle of the low frequency band, has a duration of approximately 20 to 30 seconds.

It is well known in the field of signal processing (a filed that uses spectral analytical procedures extensively) that a signal that varies cyclically over time can often best be analyzed by separating the time-varying signal into multiple waveforms, and analyzing the amplitude of each waveform which is called the frequency spectrum. In electronics, it is common to generate a frequency spectrum using analog components. The fast Fourier Transform (FFT) is a mathematical technique that may be used in digital signal processing to carry out the transformation from a time domain signal to a frequency spectrum. The present invention uses the FFT to analyze heart rate data by transforming several minutes of heart rate data into a spectral density plot (or alternatively, to a periodogram). FIG. 3 shows a typical heart rate signal, in the form of a plot of heart rate (expressed as interbeat interval, or R-R interval, which is the duration of a cardiac cycle expressed in milliseconds or seconds) with respect to time. FIG. 4 shows the results of applying the FFT algorithm to the heart rate signal of FIG. 3, and in particular the various peaks or FFT “bands.” Each FFT band reflects the intensity of fluctuations at a specific frequency. When the amplitude of heart rate oscillations within a frequency range is large, the FFT peak for that frequency band is larger. When the amplitude of heart rate oscillations within a frequency range is lower, the FFT peak for that frequency band is smaller. A discussion of using the FFT in analyzing heart rate data is found in Pomeranz et al., “Assessment of Autonomic Function in Humans by Heart Rate Spectral Analysis,” American Journal of Physiology, vol. 248, p. H151 (1985).

The frequency bands in the FFT quantify heart rate fluctuations and contain important physiological information. An interdisciplinary team at MIT used surgical procedures and pharmacological blockades to characterize the biological basis of three frequency bands, and described the results in Akselrod, S. D., et al., “Hemodynamic Regulation: Investigation By Spectral Analysis,” American Journal of Physiology, vol. 249, pp. H867-H875 (1985). Their results may be summarized as follows:

1.) The high frequency band is related to parasympathetic activity (respiratory sinus arrhythmia heart rate (RSA) or vagal tone),

2.) The intermediate frequency band is related to baroreceptor activity (Trauge-Hering-Meyer wave [THM]),

3.) The low frequency band (LFB) is related to RAAS and/or SNS activity.

The MIT team established discrete frequency bands for these three systems. The present invention utilizes the low frequency band established by the MIT team.

Most of the previous frequency spectrum analysis of heart rate signals has focused on higher frequency bands. See, e.g., Richards et al., “Heart Rate Variability During Attention Phases in Young Infants,” Psychophysiology, vol. 28, pp. 43-53 (1989). While studies carried out by the Inventor indicate that the low frequency band (LFB) may reflect neurohormonal activity involved in regulation of blood flow (i.e., vasodilation and/or vasoconstriction to different tissues or organs; blood pressure regulation) conflicting interpretations of the low frequency band have obscured its utility as an indication of blood flow changes to active tissues.

In contrast, the present invention uniquely focuses on the low frequency band, based on studies carried out by the Inventor and colleagues, one of which is described in Hyde et al., “Cardiac Rhythmicities And Attention in Young Children,” Psychophysiology, vol. 34, pp. 547-552 (1997). The Hyde et al. article compares the three frequency bands and concludes that the low frequency band best distinguishes the condition of attention from an unfocused resting condition in young children, but this article does not describe any practical application for this discovery or the means for practicing this discovery.

In summary, the elements of this patent are well known: changes in blood flow to tissues based on activity in the tissue, heart rate variability, and spectral analysis of time series data. However, there is no recognition of the general utility of heart rate oscillations as a measure of sustained vasodilation and vasoconstriction, much less the specific use of the low frequency band (LFB) of a heart rate frequency spectrum, for a physiological monitoring and diagnostic purposes. In addition, there is little recognition of the utility of analyzing heart rate variability under controlled conditions, much less under two conditions designed to specifically elicit physiological processes related to a specific medical condition or physiological function. The advantages of such analysis are that heart rate can be measured inexpensively and non-invasively and yet, for many conditions, provides unique information about the functional activation of physiological systems that cannot be obtained from conventional tests.

### SUMMARY OF THE INVENTION

It is accordingly an objective of the invention to provide a method of quantitatively analyzing changes in heart rate related to shifts in blood to tissues or organs in response to a particular activity or stimulus, and of utilizing the results of the quantitative analysis to diagnose specific medical conditions by comparing the results to the normal results obtained from healthy people collected under identical conditions. The results of the quantitative analysis may also be used to select treatment options, monitor response to treatment (i.e., titration), and side effects of treatments.

It is a second objective of the invention to provide a method of continuously and non-invasively monitoring the activity of the neurohormonal system that regulates long-term changes in blood flow to different tissues or organs.

It is a third objective of the invention to provide a method of monitoring blood flow to tissues and organs which is less expensive and can be used much more conveniently than current technologies that monitor blood flow, such as PET scanners and Doppler monitors.

It is a fourth objective of the invention to provide an apparatus for carrying out the method of the first three objectives.

It is a fifth objective of the invention to provide normative data bases of heart rate spectra that are related to, and useful for diagnosis of, specific physiological conditions.

These objectives are accomplished, in accordance with the principles of the invention, by a method of assessing physiological or mental functioning that includes the steps of: subjecting a patient to a protocol expected to activate or suppress the neurohormonal systems that regulate blood flow; in synchrony with the protocol, monitoring a physiological signal indicative of the cardiac cycle; processing the physiological signal to obtain frequency spectra during the different conditions of the protocol; comparing selected bands of the spectra to determine differences indicative of changes in blood flow; and comparing the changes in frequency spectra to predetermined normative values.

The objectives of the invention are also accomplished by apparatus for carrying out the method of the invention, including a first device for administrating a protocol; a second device for capturing a physiological signal indicative of the cardiac cycle; and a third device for analyzing the signal indicative of the cardiac cycle, wherein administration of the protocol is to capture and analysis of the physiological signal in synchrony with the events of the protocol, and wherein the third device for analyzing the physiological obtains the frequency spectra at different conditions during the protocol; comparing selected bands of the spectra to determine differences indicative of changes in blood flow; and comparing the changes in blood flow to values from a normative database.

In preferred embodiments of the method and apparatus of the invention, the protocol comprises two complementary conditions (e.g., focused mental activity versus unfocused mental activity) with instructions administered by a computer (i.e., the first device cited in the paragraph above), the physiological signal that is monitored is the electrical activity of the heart which is measured with a electrocardiograph and is recorded with an analog-to-digital board and a computer (i.e., the second device cited in the paragraph above), the heart rate time series is analyzed with a fast Fourier transform which interprets differences in the low frequency bands as an indication of person's response to the events of the protocol (i.e., the third device cited in the paragraph above). The final result of the analysis for each of the controlled conditions is the oscillation intensity in the low frequency band, which is inversely related to the activity of the neurohormonal systems that regulate blood flow. The change in oscillation intensity indicates how well the individual was able to activate or suppress the neurohormonal system during the controlled conditions, and is interpreted by comparing it to a normative database obtained from people who followed the same protocol.

More specifically, the objectives of the invention are accomplished, in accordance with the principles of a preferred embodiment of the invention, by a method including, and apparatus for performing, the steps of:
monitoring a physiological signal such as the electrical activity of the heart, while the patient performs an activity or is subjected to a particular stimulus for a predetermined period according to a protocol, capture of the physiological signal being synchronized with performance of the protocol;
mathematically processing the physiological signal, preferably by performing a fast Fourier transform (FFT) on the physiological signal;
selecting a frequency band in the FFT spectrum, said frequency band corresponding to a neurohormonal system contribution to the FFT;
comparing the change in neurohormonal activity represented by the FFT spectrum in the selected frequency band to a database of normative values.

In a preferred embodiment of the invention, the selected low frequency band is indicative of the RAAS and/or SNS response to the activity or stimulus, these systems controlling vasodilation and vasoconstriction of blood vessels that regulate blood flow to particular tissues or organs.

The “protocol” is administered by a device which a) is synchronized with the heart rate measurements and b) presents instructions that tell the subject/patient what to do. The response of the neurohormonal system is best measured when at least two conditions are measured and one of the two conditions is designed to activate the neurohormonal system and the other condition is designed to suppress the neurohormonal system (or remain in a default state). The protocol is designed to elicit a physiological response relevant to the disease, medical, mental, or physical condition of concern. Unhealthy people would be characterized by inappropriate responses (four possibilities: insufficient activation, insufficient suppression, excessive activation, excessive suppression); the magnitude of the inappropriate response would reflect the severity of the medical condition.

Heart rate is preferably measured by means of an electrocardiogram (ECG). The ECG is a very robust signal that can be reliably collected with minimal artifacts. Many other alternate physiological data indicative of heart rate could also be collected and are intended to be within the scope of the invention, but these data tend to be less reliable, i.e., they tend to have more problems with signal/noise ratio, artifacts, and loss of signal. These other physiological data include pulse measurements (which may be detected at the finger tip, ear lobe or other point); cardiac impedance, heart sounds, heart vibrations, blood pressure, Doppler measurements of heart activity or vascular activity. The electrocardiograph signal is present as an artifact in other devices such as electroencephalographs; thus, these devices can also be used to collect the heart rate signal for performing this invention.

In the preferred embodiment of the invention, physiological and protocol information is be synchronized (i.e., physiological data collected during a specific condition can be precisely identified), on the order of millisecond accuracy to obtain useful results. However, there may be situations in which less or more accurate synchronization is required.

The goal of data analysis is to answer the following question: how much did the LFB increase or decrease from Condition 1 to Condition 2. The change in the low frequency band (ΔLFB) may be interpreted as either an “appropriate” response or abnormal response to the condition. The term “appropriate” may be defined as a dichotomous measure (“within normal limits” or abnormal) or as a continuous measure (e.g., the patient has a risk level of 90% for increased blood pressure if he/she maintains a high salt diet). In either case, interpretation requires “norm tables,” which are databases containing heart rate frequency spectrum obtained by testing large numbers of individuals to determine what are normal values for the frequency spectra associated with a particular protocol. Those skilled in the art will appreciate that norm tables are a key to the evaluation of data in a variety of medical and psychological applications.

In order to obtain a useful frequency spectrum, the method of the invention may include the steps of detrending the FFT in order to remove unwanted ultra-low frequencies from the spectral plot, and use of co-variance techniques to minimize contributions of the sympathetic contributions of physiological systems other than the neurohormonal system of interest (i.e., the RAAS system). Detrending and co-variance techniques are both known method of data analysis, though not previously used for the purpose of analyzing FFT-transformed heart rate data synchronized to a patient testing protocol.

By carefully selecting the appropriate controlled conditions, the invention has, by way of example and not limitation, application to at least the following conditions: 1.) attention deficit/hyperactivity disorder; 2.) addictions including substance abuse; 3.) aggression; 4.) anxiety; 5.) other psychiatric disorders involving impulsivity and/or disordered attention, such as obsessions, compulsions, and schizophernia; 6.) autism; 7.) cardiovascular diseases such as chronic heart failure; 8.) hypertension, including salt sensitivity; 9.) spinal cord injuries; 10.) stroke; 11.) sleep disorders; 12.) toxic shock syndrome; 13.) eating disorders; 14.) obesity; 15.) metabolic disorder syndrome; 16.) sexual disorders such as erectile dysfunction; 17.) psychological disorders related to sex offenders; 18.) autonomic disorders such as Reynaud's syndrome; 19.) cardiovascular and cognitive changes associated with pregnancy; 20.) headaches; 21.) diabetes; 22.) kidney disorders; 23.) traumatic brain injury; 24.) rapidly growing tissues, such as cancer; and 25.) any other disorder or pathology that involves abnormal blood flow. In addition, the invention may also be used to evaluate non-pathological conditions such as mental effort or alertness, which are also related to changes in oscillation intensity that reflects the activation or suppression of the neurohormonal system responsible for the sustained vasodilation or vasoconstriction of cerebral blood vessels.

The invention has six applications (this paragraph defines the word “application” as used in the above paragraph) for each of the above disorders or conditions:
1.) The invention can be used to study the normal biological processes as well as the disease states that result when the underlying biological processes do not function normally; norm tables are developed from the data collected from healthy individuals.
2.) The invention can be used to assess and monitor the status of these disorders. For example, comparing an individual's results to the norm tables could be used in diagnosing a medical disorder; the invention could also be used to diagnose medical vulnerability (e.g., the likelihood of developing high blood pressure on a high salt diet); the invention could be used to also track the progression or remission of hypertension.
3.) The invention can be used to predict an individual's response to medications. For example, many of the above-identified disorders are treated by medication but predicting an individual's response to these medications is often difficult. One application of this invention is predicting which of several possible medications is best for an individual. The invention could be used identify individuals likely to have an adverse reaction to a given medication.
4.) The invention can be used to titrate medication. The disorders listed above are often treated with medication. However, determining the most appropriate dose for these medications, i.e., titrating the dosage, can be a time consuming process of trial and error. The invention can quickly assess the efficacy of various doses of medication by quantifying the effect of the medication on the oscillation intensity.
5.) The invention could also be used to monitor the effect of non-medical treatments, including but not limited to, biofeedback, relaxation, physical therapies, psychotherapy, diet, and physical exercise, in which case the invention could be used to quickly assess the efficacy of various non-medicinal treatments on the oscillation intensity.
6) The invention could be used to assess the cardiovascular side effects of various medical disease or medications. For example, children with autism may also have atypical metabolism and the metabolic byproducts adversely affect their behavior; this invention could be used to assess this side effect of the medical condition of autism. As another example, medications used to treat ADHD can affect blood pressure in some children; this invention could be used to detect children who are likely to have significant blood pressure elevations as a side effect of their medication.

### BRIEF DESCRIPTION OF THE DRAWINGS

FIG. 1 is a schematic view of a bioassessment apparatus constructed in accordance with the principles of a preferred embodiment of the invention.

FIG. 2 is a schematic diagram illustrating the use of two conditions of a protocol to obtain data that reflects changes in the neurohormonal system.

FIG. 3 is a graph of heart rate changes with time, from which the low frequency oscillation may be obtained.

FIG. 4 is a frequency spectrum of the time domain signal illustrated in FIG. 3.

FIG. 5 is flowchart illustrating the seven step data processing according to a preferred embodiment of the invention.

### DETAILED DESCRIPTION OF THE PREFERRED EMBODIMENTS

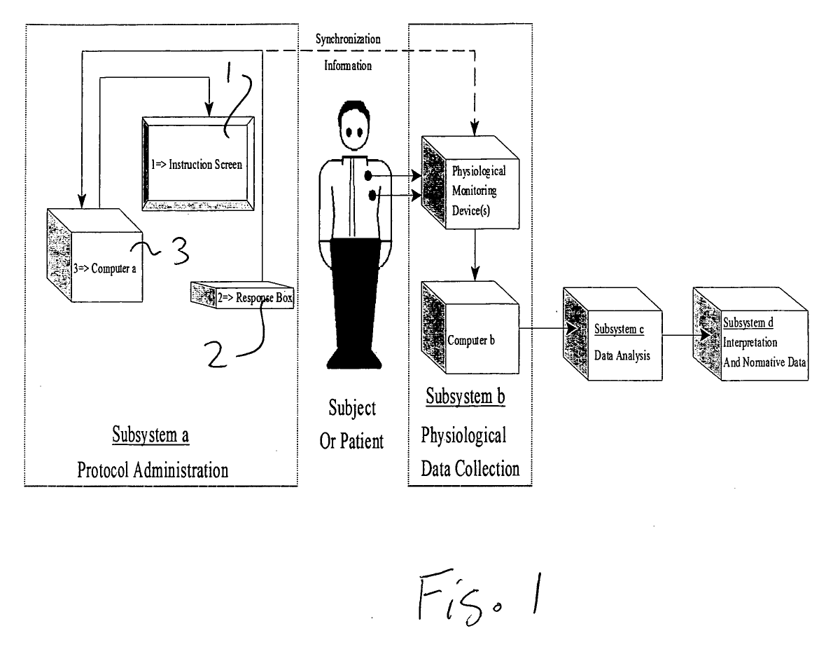

FIG. 1 shows a system according to a preferred embodiment of the invention. The illustrated system is divided into at least two subsystems: a) protocol administration and b) data collection. In addition to protocol administration and data collection, either of the illustrated subsystems or an additional subsystem may be added for data analysis (subsystem c in FIG. 1) or data interpretation (subsystem d in FIG. 1).

The protocol administration subsystem (a) administers cognitive tasks and gives instructions to the patient/subject. These functions may be done by equipment that includes an instruction screen 1 to display instructions, a response box 2 such as a touch screen, joystick, or a keypad or buttons through which the patient/subject indicates his or her responses to the instructions on the screen, and a computing device 3 for controlling the instruction screen 1 and response box 2 according to a specific protocol.

It will be appreciated that although FIG. 1 shows an instruction screen and response box, the protocol administration subsystem (a) may take any form necessary to elicit a response or administer stimuli appropriate to the condition being tested. For example, subsystem (a) may be arranged to facilitate administration of medicine or another substance such as a salt pill based on instructions presented on the instruction screen 1 or oral instructions from a technician (or both). Response box 2 may be used for the purpose of allowing a patient to manually input background information about the patient, to supply answers to questions displayed on the response screen, to input patient responses to stimuli, and so forth, or it may be omitted entirely for tests involving small children or for tests involving physical activities. In the latter case, subsystem (a) may include exercise equipment or other devices designed to cause the patient/subject to perform a the physical activity. While the protocol will in many cases be managed by a computing device, the computing device may be omitted if the protocol involves tasks that can be carried out manually, such as administration of a pill, a sleep test, or the like, though it should be understood that whether or not the protocol is completely automated, strict control of the patient's activities is required in order to elicit a physiological response relevant to the medical condition, physiological function or mental function of interest.

In contrast, the data collection subsystem (b) collects physiological and cognitive/performance measures from the patient/subject and therefore requires equipment that includes, at least, a physiological measuring device, (ECG, pulse detectors, heart sounds, etc., described further below, and analog-to-digital equipment). Physiological data collected by the data collection subsystem may include ECG, heart rate derived from ECG, pulse detectors, heart sounds, heart vibrations, and/or transcranial Doppler which measures the velocity of blood in blood vessels. Performance data may, by way of example and not limitation, include the patient/subject's response to cognitive tasks (e.g., an individuals reaction time on a cognitive test) and other measures such as physical movement (e.g., amount of fidgeting).

Data interpretation, which may be performed by either of the illustrated computers A and B or by a separate subsystem C, begins with processing the raw physiological data and performing calculations necessary to obtain a usable spectrum of the data that can be analyzed by comparison with a norm. The processing steps include operations such as conversion from ECG to a time series of interbeat intervals (IBIs, also known as R-R intervals), artifact recognition and correction, detrending the corrected data, and performing a fast Fourier Transform (FFT) on the corrected and detrended data, and interpreting the results as normal or abnormal. In a preferred embodiment of the invention, the process captures a low frequency band LFB of the patient's heart rate that reflects changes in the physiological systems that regulate the supply of blood flow to different parts of the body, though other indications of physiological response to administration of the protocol may, in certain circumstances, be used. The low frequency band is typically includes the range of approximately 0.02 to approximately 0.10 Hertz, although the exact range of frequencies chosen may vary for different groups of patients, conditions, data processing requirements, and so forth.

The term physiological response relates to the notion that the body must respond appropriately to various situations to perform specific functions. For example, the heart must beat faster when physical activity increases, such as when walking up stairs. If a person cannot increase his or her heart rate (the physiological response), then that person will have difficulty performing physical activities (the function). A physiological response can be either an activation or a suppression of a physiological system. For example, a good physiological response to eating salt is suppression of the renin-angiotensin-aldosterone system (RAAS); in contrast, an unhealthy response is little or no suppression of the RAAS system, which results in poor functioning (salt sensitive hypertension, in this case). As another example, a good physiological response to the need to pay attention is activation of the angiotensin system, while an unhealthy response is little or no activation, which is reflected in poor attention (the function) such as occurs in ADHD patients. The lack of an appropriate response correlates with deficient functioning for that particular condition. In the example of salt sensitive hypertension, insufficient RAAS suppression (physiological response) leads to hypertension when excessive salt is consumed (impaired functioning). In the example of ADHD, insufficient RAAS activation (physiological response) leads to hyperactivity, impulsivity, and inattention (impaired functioning) By carefully designing a protocol, the conditions can be controlled so that a specific function is elicited. The physiological response to the conditions can be quantified in such a way as to provide a diagnosis or assessment of a medical disorder.

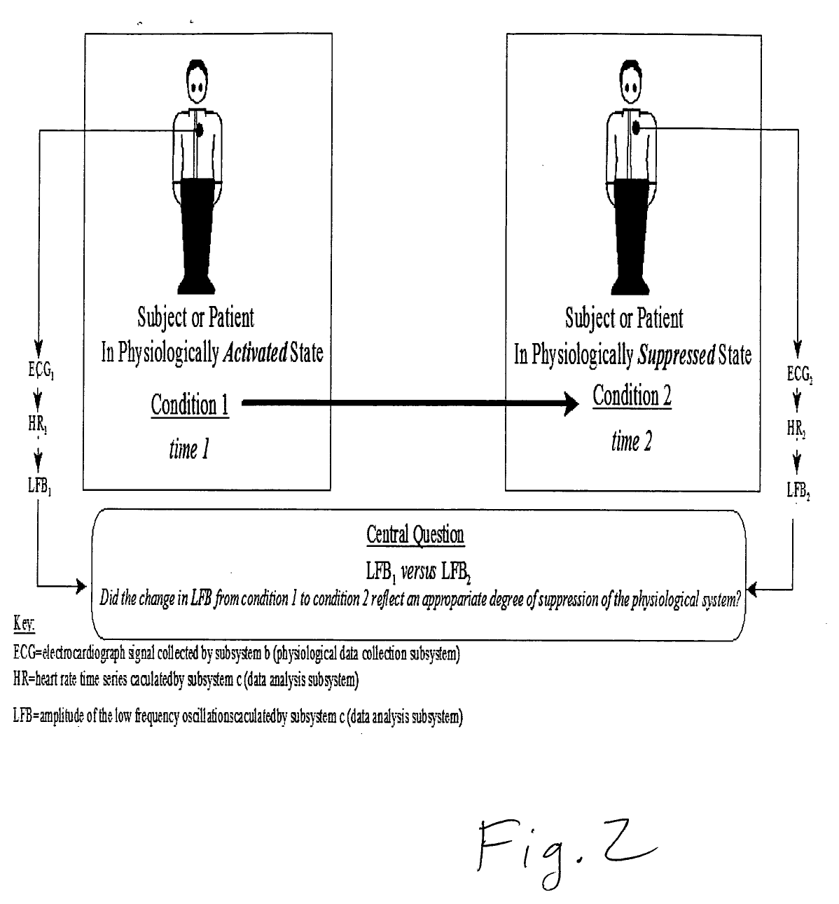

The term “protocol” as used herein and illustrated in FIG. 2 refers to what the patient does during physiological monitoring. It is not sufficient to collect these physiological measures during uncontrolled conditions, i.e., when no instructions are given to a patient/subject. The patient/subject's behavior must be carefully controlled to ensure that they follow the instructions as closely as possible. The conditions must be carefully designed to elicit the intended physiological response.

As illustrated in FIG. 2, conditions are administered in complementary pairs, with the goal being to compare physiological responses to these complementary conditions. This invention involves performing the above-described data collection while each of the conditions is administered according to the protocol. In the illustrated example, one result is a first low frequency band (LFB₁) obtained by taking an ECG during the first condition of the protocol, extracting a heart rate time series, detrending, and performing a fast Fourier Transform of the time series data. The other result is a second low frequency band (LFB₂) calculated in the same manner as the first low frequency band but based on the time series data obtained during the second condition of the protocol.

Two possible results for above data (LFB₁ versus LFB₂) are possible: LFB₁ is greater than LFB₂ or LFB₁ is less than LFB₂. The change in LFB between conditions may represent a change from activated to suppressed neurohormonal activity (or vice versa); a change from activated to default activity (or vice versa); or a change from a suppressed to default activity (or vice versa).

In the example of salt sensitive hypertension, one condition elicits suppression of a physiological response and the other condition elicits activation of a physiological response. Salt restriction in the first condition elicits angiotensin activation (which promotes salt conservation) in “healthy” individuals, while eating high salt foods elicits angiotensin suppression (promoting salt excretion) in “healthy” individuals. In the example of ADHD diagnosis, described in more detail below, focused attention elicits angiotensin activation in “healthy” individuals compared to a default level of angiotensin activity when individuals have unfocused attention. Norm tables are constructed by testing many healthy individuals under the conditions of the protocols. The physiological data from these healthy individuals define what results constitute an appropriate physiological response for a given protocol. These tables are only valid for these conditions under which they were determined.

Cognitive tasks are part of the protocol when the system is used for psychological purposes such as assessing impulsivity associated with ADHD or substance abuse, or assessing anxiety. Cognitive tasks can also be used as a part of a medical protocol, to control for mental state when measuring a physiological response for medical reasons.

It will be appreciated that there are numerous ways to measure heart rate, including electrical signals such as ECG, Holter monitor, or the cardiac signal as it appears in other methods such as EEG, EMG, etc. where it is usually considered noise (in which case the heart rate analysis provided by the invention could provide information to supplement the EEG, EMG, etc.). In addition, acoustic signals, i.e., heart sounds or related technologies; optical signals such as plethysmographs or related technologies; magnetic signals such as MEG or related technologies; or Doppler based signals such as TCD, echo cardiography or related signals; or other signals such as near infrared signals may be used to extract or derive a heart rate time series, and that any of these signals may be used as an indicator of the cardiac cycle in connection with the present invention.

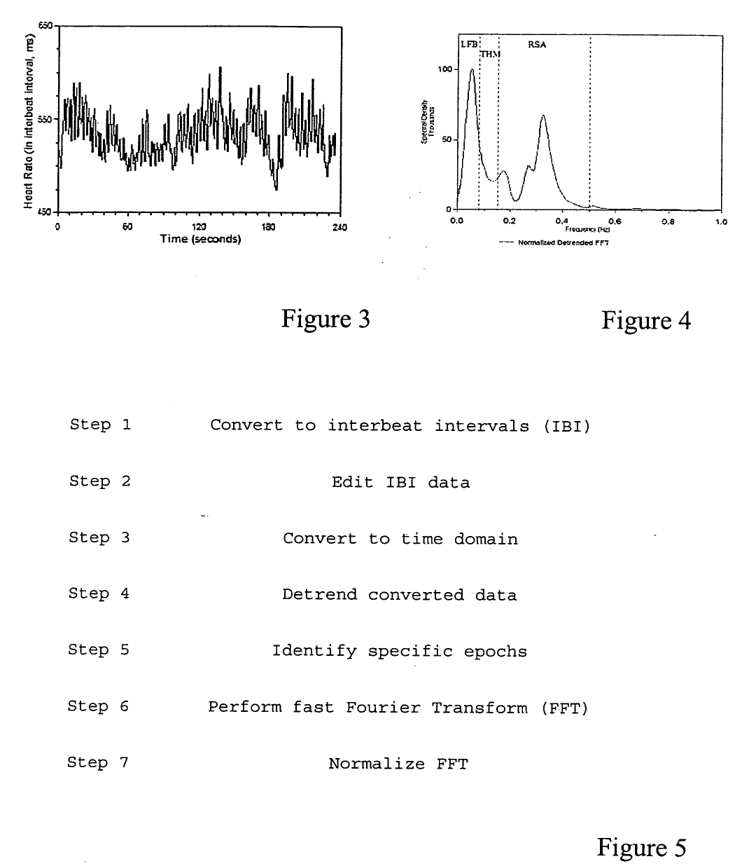

In addition, it will be appreciated that data processing will depend on the specific signal involved. However, in the illustrated embodiment of a heart rate signal illustrated in FIG. 3, physiological data reduction is preferably accomplished with an seven-step data reduction process prior to final analysis. Subsystem c uses algorithms to automate the following steps of data porcessing: In the first step, the ECG data are converted to interbeat intervals (IBI). Second, the IBI data are edited to correct artifacts (verified by comparing the heart rate to the original ECG data). Third, the data are converted from the event domain to the time domain. Fourth, the data are detrended with a numeric analytical technique known as a cubic spline approximation. The cubic spline parameters are selected to remove the undesired low frequency noise. Without detrending, the assumptions of the FFT are violated and the low frequency oscillations can be greatly overestimated. Fifth, specific conditions of the protocol are identified and, sixth, a FFT is performed with or without spectral smoothing (e.g, a nine point Hamming window). Seventh, the area under the curve is integrated for subsequent interpretation. The FFT data may be normalized as required by the specific application, or the FFT data may be used without normalization.

It will be appreciated by those skilled in the art that various mathematical algorithms may be substituted for any of the signal processing steps listed above, including digital filters such as FIR and IIR filters, and analog filters or bandpass filters. The purpose of detrending or filtering is to separate the low frequency band (LFB) from the noise-induced low frequency tail that appears in the FFT power spectrum. One way to reduce low frequency noise is simply to record data for short periods of less than three minutes, but this reduces the reliability of the measurement of the LFB. By using appropriate noise reduction algorithms or filters, it is possible to extend the data recording period for more than three minutes.

In addition to use of FFT, those skilled in the art will appreciate that frequency detection may be accomplished by alternative methods such as polynomial filtering, quantifying the RMS variation with appropriate filters, and wavelet analysis, and that all such frequency detection methods of algorithms are intended to be included within the scope of the invention.

### EXAMPLES

### Example #1

### Assessment of ADHD

This example illustrates how the system can be used as an assessment technique for ADHD. The protocol is designed to test how well the neurohormonal systems are activated when an individual attempts to pay attention for a prolonged period of time. All references to apparatus are to the apparatus illustrated in FIG. 1.

During the protocol, a computer A administers the instructions for the subject/patient. During a resting condition in which the subject/patient is instructed to not focus on any particular thoughts (i.e., to let their minds drift) for 6.5 minutes (first condition). The subject/patient sits quietly and watches an uninteresting stimulus on a computer screen during this first condition. Then in the second condition, the subject/patient performs a cognitive task which requires him or her to press a specific button depending on the stimulus that is presented on the screen (second condition). This task requires a) quickly processing information and making the appropriate response; b) being able to inhibit one's responses when required; c) sustaining attention during an uninteresting task. Performance information includes reaction time measured with millisecond accuracy, which is a measure of mental efficiency, and number of errors,which is a measure of ability to remain focused for a prolonged period of time. Those skilled in the art will appreciate that longer times provide better measures of the LFB of any signal, but that length of time is limited by the length of time for which a person will reasonably sit. 6.5 minutes is a compromise between these two competing constraints (sufficient data versus tolerable protocol) but may of course be varied depending on the protocol (for example, how fun or interesting the task) and the characteristics of the subject, such as age.

Second, for this example, computer B collects one or more channels of physiological data. ECG is preferred, as noted above, but other methods can also be used. Computer B interfaces with the physiological monitors through a known analog-to-digital conversion board.

Third, for this example, computers A and B are synchronized to within one millisecond accuracy. Digital signals are sent between computer A and computer B at specific points in the protocol to synchronize the two computers, after which the data analysis and data interpretation procedure described above (heart rate time series extraction, detrending, and FFT application) is performed.

### Example #2

### Titration of Medication for ADHD

This example illustrates how the system can be used as an titration technique for medication of ADHD. The protocol is designed to test how medication activates a neurohormonal response or, in other words, how medication facilitates the body to make the appropriate physiological response necessary for paying attention.

According to the protocol, computer A administers five conditions:

a) 6.5 minutes of unfocused mental condition, with no medication;

b) 6.5 minutes of paying attention condition, with no medication;

c) patient takes medication and waits sufficient time (commonly 60 minutes) for the medication to take effect;

d) repeat of condition A (unfocused mental condition), but now with medication active in the bloodstream; and

e) repeat of condition B (focused mental condition) but now with medication active in the bloodstream.

Data analysis and interpretation again involves the steps described above, including detrending and FFT conversion. The medicine is titrated to an optimal dose when the change in LFB from condition (d) to condition (e) is in the expected range for healthy individuals. The steps (c), (d) and (e) may be repeated with successively higher doses to determine the optimal level of medication.

### Example #3

### Side Effects of Medications for ADHD

This example illustrates how the system can be used to assess side effects of medication for ADHD. The protocol is designed to test how medication activates the cardiovascular system and blood pressure. ADHD medications increase sympathetic nervous system (SNS) activity resulting in increased blood pressure and heart rate. For most patients, these effects are nominal changes that pose little or no health risks. However, some patients have adverse effects, such as clinically elevated blood pressure. Extremely high blood pressure elevations are potentially damaging because of the long term impact of high blood pressure on nearly all the organs in the body. This application may or may not involve LFB data.

Data analysis and data interpretation is the same as before but with the addition of measuring the blood pressure before and after administering medication; unlike conventional blood pressure measurement (which typically measure blood pressure only once), hundreds of blood pressure data points are collected during the medicated and unmedicated conditions. Many data points are needed to overcome the high degree of beat-to-beat variability in blood pressure. Individuals who are vulnerable to medicine-related hypertension will have significantly elevated blood pressure after taking medication in this test.

In rare cases, a person may die while taking ADHD medication due to sudden cardiac death. The risk of cardiac sudden death is indicated by several cardiac markers, such as prolonged QT interval and decreased heart rate variability. In the case of ADHD, the data collected by this invention is intended primarily to assess the LFB as it pertains to the ability to pay attention. However, by analyzing different aspects of the ECG data, this invention can be used to assess the risk of cardiac sudden death.

Data analysis and data interpretation is the same as before but with the addition of calculating the QT interval from the ECG data to determine if a person may be susceptible to sudden cardiac death. Individuals who are vulnerable to cardiac sudden death have significantly prolonged QT interval and/or significantly reduced heart rate variability. These results could be confirmed by a closely monitored trial of medication in which the patient repeats the protocol with a dose of medication; such trials would require appropriate medical monitoring.

### Example #4

### Assessment of Salt Sensitivity

This example illustrates how the system can be used as an assessment technique for salt sensitive hypertension. The protocol is designed to test how well the neurohormonal systems are suppressed when an individual consumes salt. All references to apparatus are to the apparatus illustrated in FIG. 1.

The salt sensitivity protocol requires that individuals come to the testing session in a salt depleted state. It is not possible to become salt depleted quickly. Usually, one or more days of following a salt restricted diet are required. Individuals limit their salt intake to <500 mg sodium per day prior to the actual test. Following a salt restricted diet activates the angiotensin system.

First, the computer A administers six conditions in the salt restricted state (approximately 40 minutes of baseline data):

a.) 6.5 minutes of paced breathing (controls for respiration rate effects);

b.) 6.5 minutes of paying attention condition (controls for mental activity effects);

c.) 6.5 minutes of spontaneous breathing;

d.) Conditions a, b, and c are repeated;

e.) Patient takes salt (in the form of tablets or solution) and is monitored for the next 110 minutes to assess the effect of the salt. During this 110 minutes, conditions a, b, and c are repeated five times each.

The data is processed as described above, including detrending and FFT conversion. A large increase in LFB indicates that the RAAS has been suppressed which helps the body excrete excess sodium. RAAS suppression is healthy in this protocol because it helps to keep blood pressure in the normal, healthy range. However, people who cannot suppress their RAAS will have little or no increase in LFB. They are at higher risk for having high blood pressure and other health problems if they regularly eat a high salt diet.

### Example #5

### Titration of Medication for Blood Pressure

This example shows how the system can be used to titrate medication for treating high blood pressure, also known as hypertension. The protocol is designed to test how medication activates a neurohormonal response for maintaining healthy blood pressure or, in other words, how medication facilitates the body to make the appropriate physiological response necessary to maintain healthy blood pressure. High blood pressure is associated with excessive RAAS activity. The advantage of the system for this application is to reduce the time needed to select the optimal medication for high blood pressure and then to titrate dose of medication. Without individual information, this process can involve extensive trial-and -error. The generic term for this type of application is individualized medicine. This approach uses individual information to choose a treatment for an individual, in contrast to using population-based recommendations that may or may not be appropriate for an individual patient.

According to the blood pressure titration protocol, the patient comes for initial testing in a high blood pressure state. Computer A administers five conditions:

a.) 6.5 minutes of unfocused mental condition, no medication;

b.) 6.5 minutes of paying attention condition, no medication;

c.) Patient takes medication and waits for the medication to take effect, which may be hours or days;

d.) Condition A, unfocused mental condition, is repeated with medication in the bloodstream;

e.) Condition B, focused mental condition, is repeated with medication active in the bloodstream.

The data are processed as described above. The effectiveness of the medication will be determined by how much LFB is increased. An increase in LFB is considered a beneficial because it indicates that the medication has successfully suppressed the RAAS system.

The invention could also be used to test the effectiveness of medicine and could be used to ascertain the optimum use of various foods, such as determining what amount, if any, of salt (or cholesterol) content could have an adverse or positive effect on a user.

Having thus described a preferred embodiment of the invention and various examples in sufficient detail to enable those skilled in the art to make and use the invention, it will nevertheless be appreciated that numerous variations and modifications of the illustrated embodiment may be made without departing from the spirit of the invention, and it is intended that the invention not be limited by the above description or accompanying drawings, but that it be defined solely in accordance with the appended claims.

What is claimed is:

1. A method of biological assessment comprising the steps of a) monitoring an individual using at least one physiological signal indicative of the cardiac cycle, b) collecting the physiological signal during at least one controlled condition, c) evaluating the intensity of the low frequency oscillations in the cardiac cycles, d) utilizing said oscillation intensity as an indication of the individual's ability to activate or suppress the neurohormonal activity, and e) interpreting the oscillation intensity as normal or abnormal by comparison to normative data which were obtained from people with similar characteristics during the same controlled conditions.

2. The method of claim 1, wherein the physiological signal is the electrocardiograph (ECG) signal collected in digital form and then converted to a heart rate time series which is detrended using a cubic spline method; and the low frequency oscillations in the heart rate time series are quantified as the area under the spectral density plot between 0.02 and 0.08 Hz.

3. The method of claim 1, wherein two controlled conditions are used, each condition being three minutes or longer; the subject follows instructions and performs a specific activity in each condition; the conditions are designed to be complementary such that the subject's activities in the two conditions elicit opposing changes in the neurohormonal systems that regulate the flow of blood to active tissues in the body; the conditions are designed to elicit activity in specific tissues (e.g., regions of the brain) or organs (e.g., the brain); these two controlled conditions may be repeated several times and/or in different orders.

4. The method of claim 1, wherein the neural component of the neurohormonal system is the autonomic nervous system (which comprises the parasympathetic and the sympathetic branches) and the hormonal component of the neurohormonal system is the renin-angiotensin-aldosterone system.

5. The method of claim 1, wherein the normative data are based on the low frequency oscillation data from the same conditions obtained from a) healthy people, b) people with a specific medical condition, and/or c) people with a specific response to a treatment during the same controlled condition(s).

6. The method of claim 1, wherein the results are used to assess normal functioning or to detect abnormal functioning indicative of a medical disorder or secondary conditions associated with a medical disorder; the term “medical disorder” includes psychological and psychiatric disorders.

7. The method of claim 1, wherein the results are used to results are used to select an optimal treatment plan including type of treatment, dosage (or strength of non-medical treatment), and potential side effects of the treatment.

8. A method of biological assessment comprising a) monitoring an individual using at least one physiological signal indicative of the cardiac cycle, b) collecting the physiological signal during two or more controlled conditions in which one condition is focused mental activity elicited by a cognitive task and the other condition is an unfocused mental state, c) evaluating the intensity of the low frequency oscillation in the cardiac cycles, d) utilizing said oscillation intensity as an indication of the individual's ability to activate or suppress the neurohormonal activity, e) interpreting the oscillation intensity as normal or abnormal by comparison to normative data which were obtained from people without attention/impulse control deficits, people with attention/impulse control deficits, people with positive responses to medications for attention/impulse control deficits and people with little or no benefit from medications for attention/impulse control deficits, and f) the results are used for assessment of mental functioning including attention deficits and impulsivity; the results are used for titration of medication or other treatments for attention deficits and impulsivity; the results are used to estimate risk of side effects of medication or other treatments for attention deficits and impulsivity.

9. A method of biological assessment comprising a) monitoring an individual using at least one physiological signal indicative of the cardiac cycle, b) collecting the physiological signal on a continual basis during a specific task or job, c) evaluating the intensity of the low frequency oscillations in the cardiac cycles, d) utilizing said oscillation intensity as an indication of the individual's ability to activate or suppress the neurohormonal activity, e) interpreting the oscillation intensity as normal or abnormal by comparison to normative data which were previously obtained from the individual being monitored while performing the task, and f) the results are used for assessment of alertness, mental functioning, and/or fitness for duty as well as means of maintaining or remedying alertness required to perform said task.

10. A method of biological assessment comprising a) monitoring an individual using at least one physiological signal indicative of the cardiac cycle, b) collecting the physiological signal during two or more controlled conditions in which a substance has been restricted in one condition and another condition in which that substance is administered to the subject/patient; these conditions may be repeated several times with similar or different doses; these conditions may be repeated several times in different orders, c) evaluating the intensity of the low frequency oscillations in the cardiac cycles, d) utilizing said oscillation intensity as an indication of the individual's ability to activate or suppress the neurohormonal activity, e) interpreting the oscillation intensity as normal or abnormal by comparison to normative data which were obtained from people with positive responses to the substance and people with neutral or negative responses to the substance, and f) the results are used for assessment of quality and degree of response to said substance and/or the results are used for determining whether a treatment is needed to remedy inappropriate responses to said substance.

11. The method of claim 10, wherein the substance is salt and the results are used for the assessment and treatment of salt sensitivity.

12. The method of claim 10, wherein the substance is a medication and the results are used for selecting an optimal medication, titrating the medication to an optimal dose, and/or assessing or managing the side effects of that medication.

13. A method of biological assessment comprising a) monitoring an individual using at physiological signals indicative of cardiac intervals and blood pressure, b) collecting the physiological signals during one or more controlled conditions, c) evaluating overall heart rate variability, QT interval from ECG, pre-ejection period, and blood pressure, d) interpreting the cardiac parameters as normal or abnormal by comparison to normative data obtain from many people, and e) using the individual results to predict susceptibility to cardiovascular complications such as cardiac sudden death or hypertension.

14. The method of claim 13 in which two conditions include at least one condition in which a substance is restricted and another condition in which a substance is administered to the subject/patient; the conditions may be repeated several times and/or in different orders; the substance can be administered in various levels or doses; and the cardiac parameters are used to predict how susceptible an individual is to developing cardiovascular complications from administering or restricting the substance.

15. Apparatus for assessing physiological or mental functioning, comprising:
a) subsystem for administrating protocol(s) that provides instructions and may collect responses during conditions designed to elicit activation or suppression of the neurohormonal systems that regulate the flow of blood to active tissues in the body;
b) a subsystem for collecting physiological data indicative of the cardiac cycle in synchrony with the protocol administration;
c) a subsystem for data processing and analysis which evaluates the intensity of the low frequency oscillations in the cardiac cycles;
d) a subsystem which interprets the oscillation intensity as an indication of neurohormonal activation or suppression, and which also interprets the oscillation intensity as normal or abnormal by comparison to normative data which were obtained from people with similar characteristics during the same controlled conditions.

16. Apparatus for assessing physiological or mental functioning as claimed in claim 15, wherein the protocol administration subsystem includes a means of presenting instructions, such as a screen, and a means for the subject to make responses and record these responses, such as a one or more buttons or keys.

17. Apparatus for assessing physiological or mental functioning as claimed in claim 15, wherein the physiological data collection subsystem monitors ECG and the physiological data collection subsystem is synchronized and coordinated with the protocol system.

18. Apparatus for assessing physiological or mental functioning as claimed in claim 15, wherein the data processing subsystem includes conversion from ECG to an interbeat interval time series (IBI, also known as R-R interval), recognition and correction of artifacts and ecotopic beats; detrending the IBI times series and quantifying the intensity of the low frequency oscillations using spectral analysis (FFT).

19. Apparatus for assessing physiological or mental functioning as claimed in claim 15, wherein the data interpretation subsystem includes a normative data set of physiological results collected during equivalent conditions from appropriate and comparing individual results to this table of normative results.
